(19) United States
(12) Patent Application Publication
(10) Pub. No.: US 2020/0281937 A1
(43) Pub. Date: Sep. 10, 2020

(54) USL-311 FOR USE IN THE TREATMENT OF CANCER

(71) Applicant: Proximagen, LLC, Plymouth, MN (US)

(72) Inventor: Peter RICHARDSON, Cambridge Cambridgeshire (GB)

(73) Assignee: Proximagen, LLC, Plymouth, MN (US)

(21) Appl. No.: 16/492,305

(22) PCT Filed: Mar. 9, 2018
(86) PCT No.: PCT/GB2018/050608

§ 371 Date: Sep. 9, 2019

(30) Foreign Application Priority Data
Mar. 10, 2017 (GB) 1703907.4

(51) Int. Cl. A61K 31/551; A61P 35/00

(52) U.S. Cl. CPC A61K 31/551; A61P 35/00

(57) ABSTRACT

The invention relates to the use of the CXCR4 antagonist 6-{4-[1-(Propan-2-yl)piperidin-4-yl]-1,4-diazepan-1-yl}-N-(pyridin-4-yl)pyridine-2-carboxamide or a pharmaceutically acceptable salt thereof in the treatment of cancers of the breast, bladder, colon, rectum and liver.

### FIELD OF THE INVENTION

This invention relates to the use of CXCR4 antagonist 6-{4-[1-(Propan-2-yl)piperidin-4-yl]-1,4-diazepan-1-yl}-N-(pyridin-4-yl)pyridine-2-carboxamide in the treatment of cancers of the breast, bladder, colon, rectum and liver.

### BACKGROUND OF THE INVENTION

CXCR4 is a G-protein coupled receptor whose natural endogenous ligand is the cytokine SDF-1 (stromal derived factor-1; also referred to as CXCL12). CXCR4 was first discovered as a co-receptor, with CD4, for the entry of T-cell line-tropic (X4) HIV-1 into T-cells. CXCR4 manipulation (in combination with granulocyte colony stimulating factor (G-CSF)) has proven to improve the outcome of haematopoietic (Broxmeyer et al., 2005) and endothelial progenitor cell (Pitchford et al., 2009) stem cell mobilization. The CXCR4-SDF-1 interaction is also a master regulator of cancer stem cell trafficking in the human body (Croker and Allan, 2008) and plays a key role in the progression and metastasis of various types of cancer cells in organs that highly express SDF-1 (Zlotnik, 2008).

Several types of cancers express CXCR4 and SDF-1 which are strongly implicated in the maintenance of cancer stem cells (Wang et al., 2006; Croker and Allan, 2008) and in the recurrence of tumours after therapy. In addition CXCR4 has been shown to have a role in the formation of new blood vessels in experimental tumours (Kioi et al., 2010).

SDF-1 is a chemokine overexpressed in many tumours which activates the CXCR4 receptor located on the surface of cancer stem cells as well as many immune cells (Kumar et al., Immunity. 2006 25(2):213-24). Activation of this receptor has been implicated in the metastatic spread of many cancers (Mukherjee et al., Am J Cancer Res. 2013; 3(1): 46-57), in the formation of the tumour vasculature (Kozin et al., 2010; Kioi et al., 2010), and in both the recruitment and exclusion of immune cells from tumours (Feig et al., Proc Natl Acad Sci USA. 2013; 110(50):20212-7). It has been suggested that blockade of the CXCR4/CXCL12 axis would be beneficial in cancer treatment (Righi et al., Cancer Res. 2011; 71(16):5522-34; Vianello et al., J Immunol. 2006; 176(5):2902-14; Joyce and Fearon 2015; Richardson Anti-cancer agents in Med. Chem 2016 16(1):59-74). However other studies suggest that SDF-1 promotes immunological control of tumour growth (Nomura et al., Int J Cancer. 2001; 91(5):597-606; Fushimi et al., Cancer Res. 2006; 66(7):3513-22; Williams et al., Mol Cancer. 2010; 9:250; and Dannussi-Joannopoulos et al., Blood. 2002; 100(5):1551-8).

WO2012/049277 teaches the structure and preparation of CXCR4 antagonist 6-{4-[1-(Propan-2-yl)piperidin-4-yl]-1,4-diazepan-1-yl}-N-(pyridin-4-yl)pyridine-2-carboxamide and has the structure:

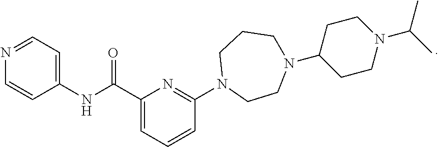 [SMILES: CC(C)N1CCC(N2CCCN(c3cccc(C(=O)Nc4ccncc4)n3)CC2)CC1]

Cancer is a major cause of death which can in some cases be cured, especially if identified early in disease development. In humans, cancers include, for example, breast, bladder, colorectal, skin, lymph, lung, kidney and liver cancer.

There is therefore the need for compounds that are effective for use in the treatment of cancer types.

### SUMMARY OF THE INVENTION

In experimental studies it has been surprisingly found that CXCR4 antagonist 6-{4-[1-(Propan-2-yl)piperidin-4-yl]-1,4-diazepan-1-yl}-N-(pyridin-4-yl)pyridine-2-carboxamide is particularly effective in inhibiting tumour growth in suitable models for particular cancer types.

Thus, a first aspect of the invention makes available 6-{4-[1-(Propan-2-yl)piperidin-4-yl]-1,4-diazepan-1-yl}-N-(pyridin-4-yl)pyridine-2-carboxamide, or a pharmaceutically acceptable salt thereof, for use in the treatment of breast, bladder, colon, rectal or liver cancer. The use is not in combination with an immune checkpoint inhibitor.

A further aspect of the invention makes available the use of 6-{4-[1-(Propan-2-yl)piperidin-4-yl]-1,4-diazepan-1-yl}-N-(pyridin-4-yl)pyridine-2-carboxamide, or a pharmaceutically acceptable salt thereof, in the manufacture of a medicament for the treatment of breast, bladder, colon, rectal or liver cancer. The use is not in combination with an immune checkpoint inhibitor.

Another aspect of the invention makes available a method of preventing or treating breast, bladder, colon, rectal or liver cancer comprising administering to a human or animal subject in need thereof 6-{4-[1-(Propan-2-yl)piperidin-4-yl]-1,4-diazepan-1-yl}-N-(pyridin-4-yl)pyridine-2-carboxamide, or a pharmaceutically acceptable salt thereof, in a sufficient amount to provide a therapeutic effect. 6-{4-[1-(Propan-2-yl)piperidin-4-yl]-1,4-diazepan-1-yl}-N-(pyridin-4-yl)pyridine-2-carboxamide is not administered in combination with an immune checkpoint inhibitor.

In an embodiment 6-{4-[1-(Propan-2-yl)piperidin-4-yl]-1,4-diazepan-1-yl}-N-(pyridin-4-yl)pyridine-2-carboxamide is the sole pharmaceutically active agent.

The cancer may be a breast cancer. The cancer may be a bladder cancer. The cancer may be a colon cancer. The cancer may be a rectal cancer.

The cancer may be a liver cancer.

Cancer cells may be eliminated. Tumour mass may be reduced.

The inventors have surprisingly found that the level of expression of the chemokine SDF-1 in cancer cells can be used to identify patients having cancer who are likely to respond to treatment with a therapeutically effective amount of 6-{4-[1-(Propan-2-yl)piperidin-4-yl]-1,4-diazepan-1-yl}-N-(pyridin-4-yl)pyridine-2-carboxamide or a pharmaceutically acceptable salt thereof.

Specifically, it was found that high levels of SDF-1 in a sample from a patient having cancer can be used to identify whether that patient will respond to treatment with 6-{4-[1-(Propan-2-yl)piperidin-4-yl]-1,4-diazepan-1-yl}-N-(pyridin-4-yl)pyridine-2-carboxamide or a pharmaceutically acceptable salt thereof.

Thus, in an embodiment the invention concerns the treatment of breast, bladder, colon, rectal or liver cancer wherein a human or animal subject having the breast, bladder, colon, rectal or liver cancer has an SDF-1 level of at least 10 FPKM.

In an embodiment the invention concerns the treatment of breast, bladder, colon, rectal or liver cancer wherein a sample from a human or animal subject having the breast, bladder, colon, rectal or liver cancer has an SDF-1 level of at least 10 FPKM.

In an embodiment, the invention concerns a method of treating or preventing a tumour and/or cancer comprising: determining whether a tissue sample from a human or animal subject has a high level of SDF-1; and selectively administering to the human or animal subject in need thereof 6-{4-[1-(Propan-2-yl)piperidin-4-yl]-1,4-diazepan-1-yl}-N-(pyridin-4-yl)pyridine-2-carboxamide, or a pharmaceutically acceptable salt thereof, in sufficient amounts to provide a therapeutic effect, based on said tissue sample having been previously determined to have an SDF-1 level of at least 10 FPKM.

The tissue sample may be a tumour or a portion thereof. A high level of SDF-1 may be at least 10 FPKM. The SDF-1 level may be at least 11 FPKM. The SDF-1 level may be at least 12 FPKM. The SDF-1 level may be at least 13 FPKM. The SDF-1 level may be at least 14 FPKM. The SDF-1 level may be at least 15 FPKM. The SDF-1 level may be at least 16 FPKM.

### DETAILED DESCRIPTION OF THE INVENTION

Any suitable form of the 6-{4-[1-(Propan-2-yl)piperidin-4-yl]-1,4-diazepan-1-yl}-N-(pyridin-4-yl)pyridine-2-carboxamide can be used. These include salts, prodrugs and active metabolites thereof. Suitable dose ranges for the 6-{4-[1-(Propan-2-yl)piperidin-4-yl]-1,4-diazepan-1-yl}-N-(pyridin-4-yl)pyridine-2-carboxamide are known in the art.

The dose of 6-{4-[1-(Propan-2-yl)piperidin-4-yl]-1,4-diazepan-1-yl}-N-(pyridin-4-yl)pyridine-2-carboxamide will of course depend on the usual factors, but is preferably at least 0.2, e.g. at least 1, and may be up to 40 or 50 mg/kg/day. In an embodiment the dose of 6-{4-[1-(Propan-2-yl)piperidin-4-yl]-1,4-diazepan-1-yl}-N-(pyridin-4-yl)pyridine-2-carboxamide is from 5 to 100 mg/day. In another embodiment the dose is from 10 to 90 mg/day. In another embodiment the dose is from 20 to 80 mg/day. In another embodiment the dose is from 30 to 70 mg/day.

The CXCR4 antagonist of the invention may be administered by any available route, such as via the oral, inhaled, intranasal, sublingual, intravenous, intramuscular, rectal, dermal, and vaginal routes. The CXCR4 antagonist is preferably administered via the oral or intravenous route. In an embodiment, the 6-{4-[1-(Propan-2-yl)piperidin-4-yl]-1,4-diazepan-1-yl}-N-(pyridin-4-yl)pyridine-2-carboxamide is administered orally or intravenously.

The 6-{4-[1-(Propan-2-yl)piperidin-4-yl]-1,4-diazepan-1-yl}-N-(pyridin-4-yl)pyridine-2-carboxamide is preferably formulated to be administered orally, for example as tablets, troches, lozenges, aqueous or oral suspensions, dispersible powders or granules. In an embodiment pharmaceutical compositions comprising 6-{4-[1-(Propan-2-yl)piperidin-4-yl]-1,4-diazepan-1-yl}-N-(pyridin-4-yl)pyridine-2-carboxamide are tablets or capsules. Liquid dispersions for oral administration may be syrups, emulsions and suspensions. Alternatively, the 6-{4-[1-(Propan-2-yl)piperidin-4-yl]-1,4-diazepan-1-yl}-N-(pyridin-4-yl)pyridine-2-carboxamide may be formulated as a pressed tablet or capsule with conventional excipients, examples of which are given below. These may be immediate release or modified, sustained or controlled release preparations.

Compositions intended for oral use may be prepared according to any method known to the art for the manufacture of pharmaceutical compositions, and such compositions may contain one or more agents selected from the group consisting of sweetening agents, flavouring agents, colouring agents and preserving agents in order to provide pharmaceutically elegant and palatable preparations. Tablets may contain the 6-{4-[1-(Propan-2-yl)piperidin-4-yl]-1,4-diazepan-1-yl}-N-(pyridin-4-yl)pyridine-2-carboxamide in admixture with non-toxic pharmaceutically acceptable excipients which are suitable for the manufacture of tablets. These excipients may include but are not restricted to, inert diluents, such as calcium carbonate, sodium carbonate, lactose, calcium phosphate or sodium phosphate; granulating and disintegrating agents, for example corn starch or alginic acid; binding agents, for example starch gelatin, acacia, microcrystalline cellulose or polyvinyl pyrrolidone; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate, or glyceryl distearate may be employed.

Aqueous suspensions may contain the 6-{4-[1-(Propan-2-yl)piperidin-4-yl]-1,4-diazepan-1-yl}-N-(pyridin-4-yl)pyridine-2-carboxamide in admixture with excipients suitable for the manufacture of aqueous suspensions. Such excipients are suspending agents, for example sodium carboxymethylcellulose, methylcellulose, hydroxypropylmethylcellulose, sodium alginate, polyvinyl pyrrolidone, gum tragacanth and gum acacia; dispersing or wetting agents may be a naturally occurring phosphatide, for example lecithin, or condensation products of an alkylene oxide with fatty acids, for example polyoxyethylene stearate, or condensation products of ethylene oxide with long-chain aliphatic alcohols, for example heptadecaethyleneoxycetanol, or condensation products of ethylene oxide with partial esters derived from fatty acids, for example polyoxyethylene sorbitan monooleate. The aqueous suspensions may also contain one or more preservatives, for example ethyl or n-propyl p-hydroxybenzoate, one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, polyoxyethylene hydrogenated castor oil, fatty acids such as oleic acid, or in a mineral oil such as liquid paraffin or in other surfactants or detergents. The oily suspensions may contain a thickening agent, for example beeswax, hard paraffin or cetyl alcohol. Sweetening agents, such as those set forth above, and flavouring agents may be added to provide a palatable oral preparation. These compositions may be preserved by the addition of an antioxidant such as ascorbic acid.

Dispersible powders and granules suitable for preparation of an aqueous suspension by the addition of water provide the combined active ingredients in admixture with a dispersing or wetting agent, suspending agent and one or more preservatives. Suitable sweetening, flavouring and colouring agents may also be present.

The pharmaceutical compositions may also be in the form of oil-in-water emulsions. The oily phase may be a vegetable oil, for example olive oil or arachis oil, or a mineral oil, for example liquid paraffin, or mixtures of these. Suitable emulsifying agents may be naturally occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative, flavouring and colouring agents. Suspensions and emulsions may contain a carrier, for example a natural gum, agar, sodium alginate, pectin, methylcellulose, carboxymethylcellulose, or polyvinyl alcohol.

In an embodiment, 6-{4-[1-(Propan-2-yl)piperidin-4-yl]-1,4-diazepan-1-yl}-N-(pyridin-4-yl)pyridine-2-carboxamide is to be administered via the oral route. Such compositions may be produced using conventional formulation techniques. In particular, spray-drying may be used to produce microparticles comprising the active agent dispersed or suspended within a material that provides the controlled release properties.

The process of milling, for example jet milling, may also be used to formulate the therapeutic composition. The manufacture of fine particles by milling can be achieved using conventional techniques. The term “milling” is used herein to refer to any mechanical process which applies sufficient force to the particles of active material to break or grind the particles down into fine particles. Various milling devices and conditions are suitable for use in the production of the compositions of the invention. The selection of appropriate milling conditions, for example, intensity of milling and duration, to provide the required degree of force, will be within the ability of the skilled person. Ball milling is a preferred method. Alternatively, a high pressure homogeniser may be used, in which a fluid containing the particles is forced through a valve at high pressure, producing conditions of high shear and turbulence. Shear forces on the particles, impacts between the particles and machine surfaces or other particles, and cavitation due to acceleration of the fluid, may all contribute to the fracture of the particles. Suitable homogenisers include the EmulsiFlex high pressure homogeniser, the Niro Soavi high pressure homogeniser and the Microfluidics Microfluidiser. The milling process can be used to provide the microparticles with mass median aerodynamic diameters as specified above. If hygroscopic, the active agent may be milled with a hydrophobic material, as stated above.

If it is required, the microparticles produced by the milling step can then be formulated with an additional excipient. This may be achieved by a spray-drying process, e.g. co-spray-drying. In this embodiment, the particles are suspended in a solvent and co-spray-dried with a solution or suspension of the additional excipient. Preferred additional excipients include polysaccharides. Additional pharmaceutically effective excipients may also be used.

Compositions intended for inhaled, topical, intranasal, intravenous, sublingual, rectal and vaginal use may be prepared according to any method known to the art for the manufacture of pharmaceutical compositions.

Therapy according to the invention may be conducted in generally known manner, depending on various factors, such as the sex, age or condition of the patient, and the existence or otherwise of one or more concomitant therapies. The patient population may be important.

In an embodiment, the invention concerns the treatment of breast, bladder, colon, rectal or liver cancer having high levels of SDF-1. Those skilled in the art know techniques and methods used for determining the level of SDF-1. For example, the SDF-1 level may be determined by performing RNA sequencing (RNA-seq). RNA-seq may be used to determine the expression of SDF-1 and may express SDF-1 expression as fragments per kilobase of exon per million reads (FPKM). A high SDF-1 level may be at least 10 FPKM. The SDF-1 level may be at least 11 FPKM. The SDF-1 level may be at least 12 FPKM. The SDF-1 level may be at least 13 FPKM. The SDF-1 level may be at least 14 FPKM. The SDF-1 level may be at least 15 FPKM. The SDF-1 level may be at least 16 FPKM.

In an embodiment the invention concerns the treatment of breast, bladder, colon, rectal or liver cancer characterised in that a therapeutically effective amount of 6-{4-[1-(Propan-2-yl)piperidin-4-yl]-1,4-diazepan-1-yl}-N-(pyridin-4-yl)pyridine-2-carboxamide, or a pharmaceutically acceptable salt thereof, is administered to a human or animal subject on the basis of the human or animal subject having an SDF-1 level of at least 10 FPKM.

In an embodiment the invention concerns the treatment of breast, bladder, colon, rectal or liver cancer characterised in that 6-{4-[1-(Propan-2-yl)piperidin-4-yl]-1,4-diazepan-1-yl}-N-(pyridin-4-yl)pyridine-2-carboxamide, or a pharmaceutically acceptable salt thereof, is administered to a human or animal subject on the basis of the human or animal subject having an SDF-1 level of at least 10 FPKM.

In an embodiment the invention concerns the treatment of breast, bladder, colon, rectal or liver cancer characterised in that a therapeutically effective amount of 6-{4-[1-(Propan-2-yl)piperidin-4-yl]-1,4-diazepan-1-yl}-N-(pyridin-4-yl)pyridine-2-carboxamide, or a pharmaceutically acceptable salt thereof, is administered to a human or animal subject on the basis of a sample from the human or animal subject having been determined to have an SDF-1 level of at least 10 FPKM.

In an embodiment the invention concerns the treatment of breast, bladder, colon, rectal or liver cancer characterised in that 6-{4-[1-(Propan-2-yl)piperidin-4-yl]-1,4-diazepan-1-yl}-N-(pyridin-4-yl)pyridine-2-carboxamide, or a pharmaceutically acceptable salt thereof, is administered to a human or animal subject on the basis of a sample from the human or animal subject having been determined to have an SDF-1 level of at least 10 FPKM.

### Terminology

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, the terms “treatment of cancer” is not intended to be an absolute term. In some aspects, the compositions and methods of the invention seek to reduce the size of a tumor or number of cancer cells, cause a cancer to go into remission, inhibit or prevent tumor growth in size or cell number of cancer cells. In some circumstances, treatment with a compound according to the claimed invention leads to an improved prognosis. Treatment as a prophylactic measure (i.e. prophylaxix) is also included. For example, a patient at risk of the occurrence or re-occurrence of cancer may be treated as described herein.

As used herein, the term “cancer” refers to the broad class of disorders characterized by hyperproliferative cell growth, either in vitro (e.g., transformed cells) or in vivo. Conditions which can be treated or prevented by the compositions and methods of the invention include, e.g., a variety of neoplasms, including benign or malignant tumours, a variety of hyperplasias, or the like. Compounds and methods of the invention can achieve the inhibition and/or reversion of undesired hyperproliferative cell growth involved in such conditions. The term “cancer” includes any solid tumor or liquid cancers, and can be metastatic or non-metastatic. Examples of cancers susceptible to treatment with the claimed compound include breast, bladder, colorectal (colon and/or rectal) and liver cancers.

As used herein, the term “tumour” is taken to mean a proliferation of heterogeneous cells, collectively forming a mass of tissue in a subject resulting from the abnormal proliferation of malignant cancer cells.

As used herein, the term “patient suffering from cancer” refers to an individual or subject that has been diagnosed with cancer or a cell proliferative disorder.

As used herein the term “therapeutic effect” means providing a therapeutic response in a subject. For example, providing a therapeutic effect includes inhibiting tumour progression or tumour growth. The skilled person understands that tumour progression in human patients can be determined by a variety of methods. For example, size of a tumour close to the skin can be measured by establishing the width and depth of the tumour with callipers, and then calculating the tumour volume. Less accessible tumours can be measured by observation of the images obtained from Magnetic Resonance Imaging (MRI) scanning. Providing a therapeutic effect also includes prolonging survival of a patient or subject beyond that expected in the absence of treatment. In an embodiment treatment of a patient or subject with a compound according to the invention prolongs survival beyond that expected in the absence of treatment by 1 or months, preferably 3 or more months, more preferably 6 or more months, yet more preferably 1 or more years, preferably 2 or more, or 3 or more, even more preferably by 5 or more years, including 10 or more years. Providing a therapeutic effect also includes eliminating cancer cells. Providing a therapeutic effect also includes tumour mass reduction.

As used herein the term “salt” includes base addition, acid addition and ammonium salts. 6-{4-[1-(Propan-2-yl)piperidin-4-yl]-1,4-diazepan-1-yl}-N-(pyridin-4-yl)pyridine-2-carboxamide is basic and so can form salts, including pharmaceutically acceptable salts with inorganic acids, e.g. with hydrohalic acids such as hydrochloric or hydrobromic acids, sulphuric acid, nitric acid or phosphoric acid and the like, and with organic acids e.g. with acetic, trifluoroacetic, tartaric, succinic, fumaric, maleic, malic, salicylic, citric, methanesulphonic, p-toluenesulphonic, benzoic, benzenesulfonic, glutamic, lactic, and mandelic acids and the like. Those compounds which have a basic nitrogen can also form quaternary ammonium salts with a pharmaceutically acceptable counter-ion such as chloride, bromide, acetate, formate, p-toluenesulfonate, succinate, hemi-succinate, naphthalene-bis sulfonate, methanesulfonate, trifluoroacetate, xinafoate, and the like. For a review on salts, see Handbook of Pharmaceutical Salts: Properties, Selection, and Use by Stahl and Wermuth (Wiley-VCH, Weinheim, Germany, 2002).

The compound “6-{4-[1-(Propan-2-yl)piperidin-4-yl]-1,4-diazepan-1-yl}-N-(pyridin-4-yl)pyridine-2-carboxamide” may exist as a solvate. The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

The compound “6-{4-[1-(Propan-2-yl)piperidin-4-yl]-1,4-diazepan-1-yl}-N-(pyridin-4-yl)pyridine-2-carboxamide” may exist in an amorphous form and/or several polymorphic forms and may be obtained in different crystal habits. Any reference herein to 6-{4-[1-(Propan-2-yl)piperidin-4-yl]-1,4-diazepan-1-yl}-N-(pyridin-4-yl)pyridine-2-carboxamide includes all forms of that compound irrespective of amorphous or polymorphic form.

In the present invention 6-{4-[1-(Propan-2-yl)piperidin-4-yl]-1,4-diazepan-1-yl}-N-(pyridin-4-yl)pyridine-2-carboxamide is not used in combination with an immune checkpoint inhibitor. This refers to the separate, simultaneous or sequential treatment of breast, bladder, colon, rectal or liver cancer with the CXCR4 antagonist of the invention and an immune checkpoint inhibitor.

As used herein the term “sole pharmaceutically active agent” means the only agent that provides a therapeutic response in a subject.

In the present invention the use is not in combination with an immune checkpoint inhibitor. Certain cells of the immune system have “checkpoint” proteins which need to be activated (or inactivated) to start an immune response. Cancer cells sometimes find ways to use these checkpoints to avoid being attacked by the host's immune system. The term “immune checkpoint inhibitor”, as used herein is an agent which targets an immune checkpoint protein, e.g., a receptor or ligand, in order to prevent deactivation of the immune system response, i.e., an immune checkpoint inhibitor inhibits a checkpoint protein.

Any immune checkpoint inhibitor is not part of the invention. The immune checkpoint inhibitor may target a checkpoint protein which may be CTLA-4, PD-1, PD-L1, PD-L2, LAG3, TIM-3, KIR, CD160, B7-H3 (CD276), BTLA (CD272), IDO (Indoleamine 2,3-dioxygenase), adenosine A2A receptor, C10ORF54, or a combination thereof. The immune checkpoint inhibitor may target a checkpoint protein selected from the group PD-L1, CTLA4, LAG 3, and KIR. The immune checkpoint inhibitor may target a ligand of a checkpoint protein which may be CTLA-4, PD-1, PD-L1, PD-L2, LAG3, TIM-3, KIR, CD160, B7-H3 (CD276), BTLA (CD272), IDO (Indoleamine 2,3-dioxygenase), adenosine A2A receptor, C10ORF54, or a combination thereof. Immune checkpoint inhibitors include biologic therapeutics, small molecules, or antibodies. The immune checkpoint inhibitor may be an antibody. For example, an immune checkpoint inhibitor can be a monoclonal antibody, a humanized antibody, a fully human antibody, a fusion protein or a combination thereof. Exemplary immune checkpoint inhibitors include antibodies selected from anti-CTLA-4, anti-PD-1, anti-PDL1, anti-PDL2, anti-LAG3, anti-TIM-3, anti-KIR, anti-CD160, anti-B7-H3 (CD276), anti-BTLA (CD272), anti-IDO (Indoleamine 2,3-dioxygenase), anti-adenosine A2A receptor, and anti-C10ORF54. Exemplary immune checkpoint inhibitors include anti-PD-1 and anti-CTLA-4 monoclonal antibodies, such as Pembrolizumab (Keytruda®), Nivolumab (Opdivo®), and Ipilimumab (Yervoy®). Exemplary immune checkpoint inhibitors include Durvalumab (MEDI4736), Atezolizumab (MPDL3280A), Avelumab (MSB0010718C), BMS936559/MDX1105, Tremelimumab, Ipilimumab, Pembrolizumab, Nivolumab, Pidilizumab, BMS986016, and Iirilumab. Immune checkpoint inhibitors may inhibit CTLA-4 or PD-1. Immune checkpoint inhibitors may inhibit PD-1. The immune checkpoint inhibitor may be an antibody selected from anti-CTLA-4, anti-PD-1, anti-PDL1, anti-PDL2, anti-LAG3, anti-TIM-3, anti-KIR, anti-CD160, anti-B7-H3 (CD276), anti-BTLA (CD272), anti-IDO (Indoleamine 2,3-dioxygenase), anti-adenosine A2A receptor, and anti-C10ORF54. The immune checkpoint inhibitor may be an anti-CTLA-4 or anti-PD-1 antibody. The immune checkpoint inhibitor may be an anti-PD-1 antibody.

### Preparation of 6-{4-[1-(Propan-2-yl)piperidin-4-yl]-1,4-diazepan-1-yl}-N-(pyridin-4-yl)pyridine-2-carboxamide

WO2012/049277 teaches the structure and preparation of CXCR4 antagonist 6-{4-[1-(Propan-2-yl)piperidin-4-yl]-1,4-diazepan-1-yl}-N-(pyridin-4-yl)pyridine-2-carboxamide, which is Example 30, and has the structure:

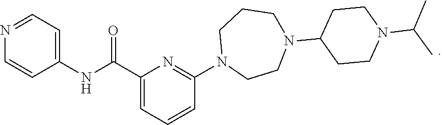 [SMILES: CC(C)N1CCC(N2CCCN(c3cccc(C(=O)Nc4ccncc4)n3)CC2)CC1]

6-{4-[1-(Propan-2-yl)piperidin-4-yl]-1,4-diazepan-1-yl}-N-(pyridin-4-yl)pyridine-2-carboxamide may be prepared using techniques known to the skilled person, including, for example, the method set out in Scheme 1.

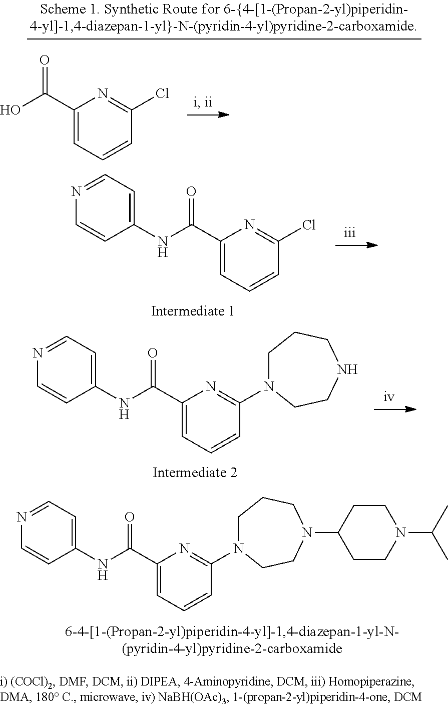

The following abbreviations have been used:

Aq aqueous

d day(s)

DCM dichloromethane

DIPEA diisopropylethylamine

DMA dimethylacetamide

DMF dimethylformamide

DMSO dimethyl sulfoxide

ES⁺ electrospray ionization

h hour(s)

HPLC High Performance Liquid Chromatography

IR Infrared Spectroscopy

LCMS Liquid Chromatography Mass Spectrometry

MeCN acetonitrile

[MH]⁺ protonated molecular ion

min minute(s)

MS Mass Spectrometry

NMR Nuclear Magnetic Spectrometry

RP reverse phase

Rt retention time

sat saturated

TFA trifluoroacetic acid

UPLC Ultra Performance Liquid Chromatography

### Experimental Methods

All reagents were commercial grade and were used as received without further purification, unless otherwise specified. Reagent grade solvents were used, unless otherwise specified. The reactions facilitated by microwave heating were performed on a Biotage Initiator system. Preparative low pressure chromatography was performed using a CombiFlash Companion or Combiflash RF systems equipped with RediSep or GraceResolv silica and C18 reverse phase columns. Preparative reverse phase HPLC was performed on a Gilson system with a UV detector equipped with a ACE-5AQ, 100×21.20 mm, 5 mm or Phenomenex Synergi Hydro-RP 80A AXIA, 100×21.20 mm, 4 mm columns. The purest fractions were collected, concentrated and dried under vacuum. Compounds were typically dried in a vacuum oven between 40° C. and 60° C. prior to purity analysis. Analytical HPLC was performed on an Agilent 1100 system. Analytical LCMS was performed on an Agilent 1100 HPLC system with a Waters ZQ mass spectrometer. NMR was performed on a Bruker Avance 500 MHz Cryo Ultrashield with Dual CryoProbe. IR analysis was performed on a Perkin Elmer FT-IR Spectrum BX using a Pike MIRacle single reflection ATR. Melting point determination was performed on a Reichert Thermovar hotstage microscope. Reactions were performed at room temperature unless otherwise stated. The compounds were automatically named using IUPAC rules.

### Intermediate 1

### 6-Chloro-N-(pyridin-4-yl)pyridine-2-carboxamide

6-Chloropyridine-2-carboxylic acid (5.50 g, 34.9 mmol) and DMF (0.5 mL) were dissolved in DCM (100 mL) and oxalyl chloride (7.09 mL, 83.8 mmol) was added. The reaction mixture was stirred for 0.5 h then the solvents were removed in vacuo. The residue was dissolved in DCM (100 mL) cooled to 0° C. DIPEA (14.6 mL, 83.8 mmol) and 4-aminopyridine (3.94 g, 41.9 mmol) were added and the reaction was allowed to warm to room temperature then stirred for a further 0.5 h. The solvents were removed in vacuo and the residue was partitioned between DCM (100 mL) and water (75 mL). The aqueous layer was extracted with DCM (2×75 mL), the organic layers combined, washed with Na₂CO₃ (1M, 75 mL), brine (75 mL), dried (MgSO₄) and the solvents removed in vacuo. The residue was purified by column chromatography to give the title compound (6.66 g, 81.7%) as an off white solid. LCMS (ES⁺): 234.2 [MH]⁺.

### Intermediate 2

### 6-(1,4-Diazepan-1-yl)-N-(pyridin-4-yl)pyridine-2-carboxamide

Intermediate 1 (1.5 g, 6.42 mmol) was dissolved in DMA (12.5 mL). Homopiperazine (3.22 g, 32.1 mmol) was added and the reaction mixture was heated using a Biotage microwave at 180° C. for 0.5 h. This process was repeated three further times on the same scale and the four batches were combined and the solvent removed in vacuo. The residue was dissolved in DCM (300 mL) and washed with sat aq Na₂CO₃ solution (150 mL), brine (100 mL), dried (MgSO₄) and the solvents were removed in vacuo. The residue was purified by column chromatography to give the title compound (6.88 g, 90.1%) as light yellow solid. LCMS (ES⁺): 298.2 [MH]⁺.

### 6-{4-[1-(Propan-2-yl)piperidin-4-yl]-1,4-diazepan-1-yl}-N-(pyridin-4-yl)pyridine-2-carboxamide

Intermediate 2 (4.88 g, 16.4 mmol) was dissolved in DCM (200 mL). 1-(Propan-2-yl)piperidin-4-one (4.88 mL, 32.8 mmol) and sodium triacetoxyborohydride (17.4 g, 82.1 mmol) were added and the reaction mixture stirred for 20 h. The reaction mixture was diluted with DCM (200 mL) and quenched with sat aq Na₂CO₃ solution (100 mL). The aqueous layer was extracted with DCM (100 mL). The organic layers were combined, washed with brine (50 mL), dried (MgSO₄) and the solvents removed in vacuo. The residue was purified by crystallisation from MeCN followed by reverse phase column chromatography. The residue was partitioned between DCM (300 mL) and sat aq Na₂CO₃ solution (100 mL). The aqueous layer was extracted with DCM (50 mL) and the organic layers were combined, washed with brine (50 mL), dried (MgSO₄) and the solvents removed in vacuo. The residue was crystallised from MeCN to give the title compound (4.66 g, 67.3%) as a light yellow solid.

HPLC: Rt 3.47 min, 100% purity

LCMS (ES⁺): 423.2 [MH]⁺

¹H NMR (500 MHz, DMSO-d₆) δ_{H} 10.31 (1H, s, NH), 8.52-8.50 (2H, m, ArH), 7.84-7.82 (2H, m, ArH), 7.70 (1H, dd, J 8.5 and 7.3 Hz, ArH), 7.30 (1H, d, J 7.2 Hz, ArH), 6.93 (1H, d, J 8.7 Hz, ArH), 3.80 (2H, m, NCH₂), 3.76 (2H, m, NCH₂), 2.82-2.79 (2H, m, NCH₂), 2.77-2.73 (2H, m, NCH₂), 2.62 (1H, spt, J 6.6 Hz, CHMe), 2.58-2.56 (2H, m, NCH₂), 2.39-2.33 (1H, m, NCHCH₂), 2.05-1.88 (2H, m, NCH₂), 1.85-1.78 (2H, m, CH₂), 1.65-1.60 (2H, m, NCHCH₂), 1.36 (2H, qd, J 11.7 and 3.4 Hz, NCHCH₂), 0.91 (6H, d, J 6.6 Hz, CH(CH₃)₂)

IR (solid) νₘₐₓ/cm⁻¹ 3328, 2936, 2358, 2162, 1982, 1682, 1597, 1582, 1510, 1485, 1459, 1418, 1404, 1383, 1364, 1336, 1282, 1246, 1211, 1179, 1161, 1125, 1070, 1030, 994, 972, 926, 898, 878, 824, 814, 758, 681 and 617

Melting point: 157-159° C.

The present invention is based at least in part on the following in vivo study.

### Study 1

Nine syngeneic cell lines from ten different cancer types (EMT-6 (breast cancer), MBT2 (bladder cancer), CT26 (colorectal cancer), B16F10small, B16BL6 (both melanoma), A20 (lymphoma), LL/2 (lung cancer), Renca (kidney cancer), H22 (liver cancer)) were cultured and when in exponential growth were inoculated in mice subcutaneously with tumour cells in 0.1 mL of PBS for tumour development. After the mean tumour size reached approximately 80-120 mm³ the mice were treated with 6-{4-[1-(Propan-2-yl)piperidin-4-yl]-1,4-diazepan-1-yl}-N-(pyridin-4-yl)pyridine-2-carboxamide (50 mg/kg p.o. 5 days out of 7). Tumour volumes were measured twice weekly at least in two dimensions using a caliper, and the volume expressed in mm³ using the formula: V=0.5 a×b² where a and b are the long and short diameters of the tumour, respectively. Tumour growth was measured and inhibition of tumour growth reported in comparison to a vehicle treated group. All groups contained 8 mice. If the tumours in a group reached an average volume of 2000 mm³, the experiment was terminated.

The results are shown in Table 1.

TABLE 1

| | | SDF-1 | % inhibition of |
|---|---|---|---|
| Tumour | | level | tumour growth |
| type | Cell line | (FPKM) | relative to control |
| Breast | EMT6 | 42 | 79.8 |
| Bladder | MBT2 | 16 | 29.3 |
| Colorectal | CT26 | 17 | 32.1 |
| Melanoma | B16F10small | 4 | 0 |
| Melanoma | B16BL6 | 2.8 | 0 |
| Lymphoma | A20 | 8 | 11.6 |
| Lung | LL/2 | 2.2 | 7.8 |
| Kidney | Renca | 3 | 0 |
| Liver | H22 | 26 | 20.1 |

Analysis of the data revealed a surprisingly selective effect of 6-{4-[1-(Propan-2-yl)piperidin-4-yl]-1,4-diazepan-1-yl}-N-(pyridin-4-yl)pyridine-2-carboxamide in significantly inhibiting the growth of breast, bladder, colon, rectal and liver tumours.

1. 6-{4-[1-(Propan-2-yl)piperidin-4-yl]-1,4-diazepan-1-yl}-N-(pyridin-4-yl)pyridine-2-carboxamide, or a pharmaceutically acceptable salt thereof, for use in the treatment of breast, bladder, colon, rectal or liver cancer;
provided that the use is not in combination with an immune checkpoint inhibitor.

2. The compound for use according to claim 1 wherein 6-{4-[1-(Propan-2-yl)piperidin-4-yl]-1,4-diazepan-1-yl}-N-(pyridin-4-yl)pyridine-2-carboxamide is the sole pharmaceutically active agent.

3. The use of 6-{4-[1-(Propan-2-yl)piperidin-4-yl]-1,4-diazepan-1-yl}-N-(pyridin-4-yl)pyridine-2-carboxamide, or a pharmaceutically acceptable salt thereof, in the manufacture of a medicament for the treatment of breast, bladder, colon, rectal or liver cancer;
provided that the use is not in combination with an immune checkpoint inhibitor.

4. The use according to claim 3 wherein 6-{4-[1-(Propan-2-yl)piperidin-4-yl]-1,4-diazepan-1-yl}-N-(pyridin-4-yl)pyridine-2-carboxamide is the sole pharmaceutically active agent.

5. A method of preventing or treating breast, bladder, colon, rectal or liver cancer comprising administering to a human or animal subject in need thereof 6-{4-[1-(Propan-2-yl)piperidin-4-yl]-1,4-diazepan-1-yl}-N-(pyridin-4-yl)pyridine-2-carboxamide, or a pharmaceutically acceptable salt thereof, in a sufficient amount to provide a therapeutic effect;
provided that the 6-{4-[1-(Propan-2-yl)piperidin-4-yl]-1,4-diazepan-1-yl}-N-(pyridin-4-yl)pyridine-2-carboxamide is not administered in combination with an immune checkpoint inhibitor.

6. The method according to claim 5 wherein 6-{4-[1-(Propan-2-yl)piperidin-4-yl]-1,4-diazepan-1-yl}-N-(pyridin-4-yl)pyridine-2-carboxamide is the sole pharmaceutically active agent administered to the human or animal subject.

7. The compound for use, use, or method according to claim 1 wherein the cancer is a breast cancer.

8. The compound for use, use, or method according to claim 1 wherein the cancer is a bladder cancer.

9. The compound for use, use, or method according to claim 1 wherein the cancer is a colon cancer.

10. The compound for use, use, or method according to claim 1 wherein the cancer is a rectal cancer.

11. The compound for use, use, or method according to claim 1 wherein the cancer is a liver cancer.

12. The compound for use, use, or method according to claim 1 wherein cancer cells are eliminated.

13. The compound for use, use, or method according to claim 1, wherein tumour mass is reduced.

14. The compound for use, use, or method according to claim 1 wherein a human or animal subject having the breast, bladder, colon, rectal or liver cancer has an SDF-1 level of at least 10 FPKM.

15. The compound for use, use, or method according to claim 1 wherein a sample from a human or animal subject having the breast, bladder, colon, rectal or liver cancer has an SDF-1 level of at least 10 FPKM.

16. The compound for use, use, or method according to claim 14 wherein the SDF-1 level is at least 11 FPKM.

17. The compound for use, use, or method according to claim 14 wherein the SDF-1 level is at least 12 FPKM.

18. The compound for use, use, or method according to claim 14 wherein the SDF-1 level is at least 13 FPKM.

19. The compound for use, use, or method according to claim 14 wherein the SDF-1 level is at least 14 FPKM.

20. The compound for use, use, or method according to claim 14 wherein the SDF-1 level is at least 15 FPKM.

21. The compound for use, use, or method according to claim 14 wherein the SDF-1 level is at least 16 FPKM.

22. A method of treating or preventing a tumour and/or cancer comprising: determining whether a tissue sample from a human or animal subject has a high level of SDF-1; and selectively administering to the human or animal subject in need thereof 6-{4-[1-(Propan-2-yl)piperidin-4-yl]-1,4-diazepan-1-yl}-N-(pyridin-4-yl)pyridine-2-carboxamide, or a pharmaceutically acceptable salt thereof, in sufficient amounts to provide a therapeutic effect, based on said tissue sample having been previously determined to have an SDF-1 level of at least 10 FPKM.

23. The method according to claim 22 wherein the SDF-1 level is at least 11 FPKM.

24. The method according to claim 22 wherein the SDF-1 level is at least 12 FPKM.

25. The method according to claim 22 wherein the SDF-1 level is at least 13 FPKM.

26. The method according to claim 22 wherein the SDF-1 level is at least 14 FPKM.

27. The method according to claim 22 wherein the SDF-1 level is at least 15 FPKM.

28. The method according to claim 22 wherein the SDF-1 level is at least 16 FPKM.

29. The method according to claim 22 wherein the cancer is breast, bladder, colon, rectal or liver cancer.
